(19) United States
(12) Patent Application Publication
(10) Pub. No.: US 2003/0022811 A1
(43) Pub. Date: Jan. 30, 2003

(54) Favorable modulation of health-related quality of life and health-related quality-adjusted time-to-progression of disease in patients with prostate cancer

(76) Inventors: Amitabh Singh, Gurnee, IL (US); Robert J. Padley, Lake Bluff, IL (US); Talat Ashraf, Vernon Hills, IL (US)

Correspondence Address: Steven F. Weinstock, Abbott Laboratories, Department 377 / AP6D-2, 100 Abbott Park Road, Abbott Park, IL, 60064-6050, US

(21) Appl. No.: 09/832,752

(22) Filed: Apr. 11, 2001

(51) Int. Cl. A61K 31/00

(52) U.S. Cl. 514/1

(57) ABSTRACT

Disclosed herein is a method for favorably modulating the health-related quality of life and the health-related quality-adjusted time-to-disease progression in a patient having prostate cancer and a method for measuring of the health-related quality-adjusted time-to-disease progression.

### TECHNICAL FIELD

This invention is directed to a method for favorably modulating the health-related quality of life and the health-related quality-adjusted time-to-disease progression in a patient having prostate cancer and a method for measuring of the health-related quality-adjusted time-to-disease progression.

### BACKGROUND OF THE INVENTION

Prostate cancer patients often face poor prognosis, limited treatment options, and a decline in their health-related quality of life (QoL) with disease progression. Because conventional analyses of responses in prostate cancer trials fail to account for effect of treatment on a patient's self-perception of their health status and general well-being, qualitative and quantitative evaluation of the multidimensional health-related QoL responses of the patient over time could potentially provide a more comprehensive assessment and understanding of the benefit of a given therapeutic intervention.

Thus, there is a long-standing need in the art for a method of favorably modulating the health-related QoL and the health-related quality-adjusted time-to-progression (QATTP) of disease in patients with prostate cancer and a method for measuring the health-related QATTP in patients undergoing treatment for prostate cancer.

### DISCLOSURE OF THE INVENTION

This invention, therefore, is directed to a method for favorably modulating the health-related quality of life and the health-related quality-adjusted time-to-disease progression in a patient having prostate cancer and a method for measuring of the health-related quality-adjusted time-to-disease progression.

The term “favorably modulating” means sustaining and/or improving the health-related QoL and sustaining and/or improving and/or extending the health-related QATTP in a patient with prostate cancer.

The term “quality-adjusted time-to-progression” or “QATTP” means the interval from the initiation of therapy to the time of documented clinical disease progression adjusted by the patient's health-related QoL score.

The term “quality of life” or “QoL” means multidimensional aspects, subjectively assessed by the patient, comprising physical functioning, emotional functioning, social/family functioning, role functioning, cognitive functioning, self-perception, and other domains relating to prostatic cancer patients such as pain, fatigue, nausea and vomiting, change in appetite, dyspnea, sleep disturbance, diarrhea, constipation, urinary function, and change in weight.

In a first embodiment for the practice of this invention, then, is a method for favorably modulating the health-related QoL of a patient with prostate cancer, preferably hormone refractory prostate cancer (HRPCa), comprising administering to the patient a therapeutically effective amount of an endothelin (ET) receptor antagonist, preferably atrasentan.

In another part of the first embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the first embodiment for the practice of this invention, the ET receptor antagonist is administered continuously.

In a preferred first embodiment for the practice of this invention is a method for sustaining the health-related QoL of a patient with prostate cancer, particularly HRPCa, comprising administering to the patient a therapeutically effective amount of an ET receptor antagonist, preferably atrasentan.

In another part of the preferred first embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the preferred first embodiment for the practice of this invention, the ET receptor antagonist is administered continuously.

In a more preferred first embodiment for the practice of this invention is a method for improving the health-related QoL of a patient with prostate cancer, particularly HRPCa, comprising administering to the patient a therapeutically effective amount of an ET receptor antagonist, preferably atrasentan.

In another part of the more preferred first embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the more preferred first embodiment for the practice of this invention, the endothelin receptor antagonist is administered continuously.

In a second embodiment for the practice of this invention, is a method for favorably modulating the health-related QATTP of a patient with prostate cancer, particularly HRPCa, comprising administering to the patient a therapeutically effective amount of an endothelin (ET) receptor antagonist, preferably atrasentan.

In another part of the second embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the second embodiment for the practice of this invention, the ET receptor antagonist is administered continuously.

In a preferred second embodiment for the practice of this invention is a method for sustaining the health-related QATTP of a patient with prostate cancer, particularly HRPCa, comprising administering to the patient a therapeutically effective amount of an ET receptor antagonist, preferably atrasentan.

In another part of the preferred second embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the preferred second embodiment for the practice of this invention, the ET receptor antagonist is administered continuously.

In a more preferred second embodiment for the practice of this invention is a method for increasing the health-related QATTP of a patient with prostate cancer, particularly HRPCa, comprising administering to the patient a therapeutically effective amount of an ET receptor antagonist, preferably atrasentan.

In another part of the more preferred second embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the more preferred second embodiment for the practice of this invention, the endothelin receptor antagonist is administered continuously.

In a third embodiment for the practice of this invention is a method for extending the health-related QATTP of a patient with prostate cancer, particularly HRPCa, comprising administering to the patient a therapeutically effective amount of an endothelin (ET) receptor antagonist, preferably atrasentan.

In another part of the third embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the third embodiment for the practice of this invention, the ET receptor antagonist is administered continuously.

In a preferred third embodiment for the practice of this invention is a method for sustaining the health-related QATTP of a patient with prostate cancer, particularly HRPCa, comprising administering to the patient a therapeutically effective amount of an ET receptor antagonist, preferably atrasentan.

In another part of the preferred third embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the preferred third embodiment for the practice of this invention, the ET receptor antagonist is administered continuously.

In a more preferred third embodiment for the practice of this invention is a method for increasing the health-related QATTP of a patient with prostate cancer, particularly HRPCa, comprising administering to the patient a therapeutically effective amount of an ET receptor antagonist, preferably atrasentan.

In another part of the more preferred third embodiment for the practice of this invention, the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still another part of the more preferred third embodiment for the practice of this invention, the endothelin receptor antagonist is administered continuously.

In a fourth embodiment for the practice of this invention is a method for determining modulation of the health-related QATTP in a patient undergoing endothelin antagonist treatment for prostate cancer, the method comprising the steps of:

(a) providing a statistically significant patient population;

(b) administering to each member of the statistically significant patient population either a therapeutically acceptable amount of an ET receptor antagonist or placebo;

(c) measuring the health-related QoL of each patient over a period of time to provide a health-related QATTP for each patient in the statistically significant patient population; and

(d) determining the sum of the mean and median health-related QATTP's for the statistically significant patient population to determine the modulation of the health-related QATTP.

In one part of the fourth embodiment, for the practice of step (a), the significant patient population comprises at least one randomized patient with prostate cancer, preferably a plurality of randomized patients with prostate cancer, more preferably a plurality of randomized patients with prostate cancer which comprises essentially about 100 patients, still more preferably a plurality of randomized patients with prostate cancer which comprises essentially about 150 patients, still even more preferably a plurality of randomized patients with prostate cancer which comprises essentially about 280 patients.

In another part of the fourth embodiment, for the practice of step (a), the prostate cancer is HRPCa.

In another part of the fourth embodiment, for the practice of step (b), the ET receptor antagonist is useful for favorably modulating, preferably sustaining, more preferably improving, the health-related QoL of the patients with prostate cancer.

In still yet another part of the fourth embodiment, for the practice of step (b), the ET receptor antagonist is useful for favorably modulating, preferably sustaining and/or extending, more preferably improving and/or extending the health-related QATTP of the patients with prostate cancer.

In still yet even another part of the fourth embodiment, for the practice of step (b), the therapeutically effective amount of the ET receptor antagonist is between about 0.01 mg per day to about 100 mg per day, preferably between about 1 mg per day to about 25 mg per day, more preferably either about 2.5 mg or about 10 mg per day.

In still yet even another part of the fourth embodiment, for the practice of step (b), the endothelin receptor antagonist is atrasentan.

In still yet another preferred part of fourth embodiment, for the practice of step (c), the period of time comprises measuring the health-related QoL of each patient at the beginning and end of the treatment session and at at least one interval time period between the beginning and the end of the treatment session, preferably about five to about seven weeks after the beginning of the treatment session, more preferably about six weeks after the beginning of the treatment session.

In a fifth embodiment for the practice of this invention is disclosed an ET receptor antagonist useful for favorably modulating, preferably sustaining and, more preferably improving, the health-related QoL of a patient with prostate cancer, particularly HRPCa.

In a sixth embodiment for the practice of this invention is disclosed an ET receptor antagonist useful for favorably modulating, preferably sustaining and/or extending, more preferably improving and/or extending, the health-related QATTP of a patient with prostate cancer, particularly HRPCa.

The ET receptor antagonists of this invention which are useful for favorably modulating the health-related QoL and/or the health-related QATTP of a patient with prostate cancer can be used in the form of therapeutically acceptable, water or oil-soluble or dispersible, acid salts. These salts include zwitterions as well as acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, lactate, maleate, methanesulfonate, nicotinate, oxalate, pamoate, pectinate, persulfate, 3-phenylpropionate, phosphoric, picrate, pivalate, propionate, succinate, sulfuric, tartrate, thiocyanate, para-toluenesulfonate undecanoate, and the like salts. Also, any basic nitrogen-containing groups can be quatemized with alkyl halides, such as methyl, ethyl, propyl, and butyl chloride, bromides, and iodides; dialkyl sulfates such as dimethyl, diethyl, dibutyl, and diamyl sulfates; long chain halides such as decyl, lauryl, myristyl and stearyl chlorides, bromides and iodides and the like.

The ET receptor antagonists of this invention which are useful for favorably modulating the health-related QoL and/or the health-related QATTP of a patient with prostate cancer can also be used in the form of therapeutically acceptable, water or oil-soluble or dispersible, basic addition salts. These salts include the hydroxide, carbonate, bicarbonate, and the like of a therapeutically acceptable metal cation or ammonia or an organic primary, secondary or tertiary amine.

The total daily dose of The ET receptor antagonists of this invention which are useful for favorably modulating the health-related QoL and/or the health-related QATTP of a patient with prostate cancer can be administered to a patient in single or divided doses in amounts such as from about 0.001 to about 1000 mg/kg body weight, usually about 0.1 to about 100 mg/kg for oral administration and about 0.01 to 10 mg/kg for parenteral administration. Dosage unit compositions may contain such amounts of submultiples thereof to make up the daily dose.

Pharmaceutical formulations comprising the ET receptor antagonists of this invention which are useful for favorably modulating the health-related QoL and/or the health-related QATTP of a patient with prostate cancer can be prepared by known procedures. The amount of active ingredient which can be combined with the carrier materials to produce a single dosage form will vary depending upon the host treated and the particular mode of administration.

It is to be understood that the specific dose level for any particular patient will depend upon a variety of factors including the activity of the compound employed, the age, body weight, general health, diet, time of administration, route of administration, rate of excretion, drug combination, and the severity of the disease.

The ET antagonists of this invention can be administered orally, buccally, parenterally, sublingually, nasally, rectally, and topically in dosage unit formulations containing conventional nontoxic pharmaceutically acceptable carriers, adjuvants, and vehicles. Topical administration includes transdermal administration such as transdermal patches or iontophoresis devices. The term parenteral means subcutaneous, intravenous, intramuscular, intrasternal, transcutaneous, and intradermal.

Injectable preparations such as sterile injectable aqueous or oleagenous suspensions can be formulated using suitable dispersing, wetting and suspending agents. The sterile injectable preparation can be a sterile injectable solution or suspension in a nontoxic parenterally acceptable diluent or solvent. Among the acceptable vehicles which can be used are water, Ringer's solution, isotonic sodium chloride solution, and sterile, fixed oils.

Suppositories for rectal administration of the drug can be prepared by mixing the drug with a suitable nonirritating excipient which is a solid at ambient temperature but a liquid at rectal temperature and will therefore melt and release the drug.

Solid dosage forms for oral administration include capsules, tablets, pills, powders, and granules. In such solid dosage forms, the active compound is admixed with an inert diluent such as sucrose or starch. These dosage forms can also comprise lubricating agents. In the case of capsules, tablets, and pills, the dosage forms can also comprise buffering agents. Tablets and pills can additionally be prepared with enteric coatings.

Liquid dosage forms for oral administration include therapeutically acceptable emulsions, solutions, suspensions, syrups, and elixirs containing inert diluents such as water. Such compositions can also comprise adjuvants, such as wetting agents, emulsifying and suspending agents, and sweetening, flavoring, and perfuming agents.

The ET receptor antagonists of this invention can also be administered in the form of liposomes. Liposomes, both natural and synthetic, are generally derived from phospholipids or other lipid substances and are formed by mono- or multi-lamellar hydrated liquid crystals dispersed in an aqueous medium. Any non-toxic, physiologically acceptable and metabolizable lipid capable of forming liposomes can be used. Liposomal compositions can contain, in addition to an ET receptor antagonist, stabilizers, preservatives, and excipients.

The ET receptor antagonists of this invention can be administered as the sole active agent or they can also be used co-therapeutically with one or more anticancer drugs or methods such as hormonal agents such as leuprolide (Lupron®); gonadorelin antagonists such as goserelin (Zoladex®) and abarelix; bicalutamide; nilutamide; flutamide; vitamin D; vitamin D analogues; estrogen and estrogen analogues such as diethylstibestrol; prednisone; hydrocortisone; ketoconazole; cyproterone acetate; progesterone; 5-alpha reductase inhibitors such as finasteride; bone-seeking radionuclides such as samarium (Quadramet®), strontium (Metastrone®), and ¹⁸⁶rhenium; external beam radiation such as three dimensional conformal radiation; brachytherapy (the implantation of radioactive seeds in the prostate); monoclonal antibodies such as trastuzumab (Herceptin®); anti-angiogenic drugs such as thrombospondin peptide or kringle 5; matrix metalloproteinase inhibitors; farnesyl transferase inhibitors; lycopenes; urokinase; plasminogen activator inhibitors; plasminogen activator receptor blockers; apoptosis inducers; selective and non-selective alpha blockers; platinum agents such as cis-platinum and carbo-platinum; taxane class drugs such as docitaxil and paclitaxil; estramustine; gemcytabine; adriamycin; doxorubicin; daunorubicin; mitoxantrone; vinblastine; vincristine; capecitabine; irinotecan; topotecan;5-fluorouracil; interferons; cytoxan; methotrexate; cytokines such as IL-2; PPAR agonists such as thiazolidine diones; retinoid-type agents, 5-lipooxygenase inhibitors such as zyflo (Zilueton®); COX-2 inhibitors; gene-therapy based therapeutics, including sense and anti-sense genes; cholesterol lowering drugs such as lovastatin, pravastatin, and simvistatin; bisphosphonates; osteoprotegrin; antibodies, both monoclonal and polyclonal; antibody-coupled radionucleotides; antibody-coupled cytotoxic agents; antibody-coupled radionucleotides; viral-vector delivered agents; vaccines directed at protein, carbohydrate, or nucleic acid targets; aminoglutethimide; and suramin.

These combinations can be administered separately or as a single dosage form containing both or all drugs. When administered as a combination, the drugs can be formulated as separate compositions, given at the same time or different times, or the therapeutic agents given as a single composition.

In addition, the ET receptor antagonists of this invention can be used in combination with one or more drugs which impede net bone loss such as estrogens, bisphosphonates, and estrogen receptor modulators, such as raloxifene and calcitonin.

The ET receptor antagonists of this invention can additionally be administered in combination with surgery such as radical prostatectomy, cryotherapy, transurethral resection of the prostate as an adjuvant, or prior to surgery as a neoadjuvant agent.

Preferred ET receptor antagonists useful for the practice of this invention are recited in commonly owned, pending U.S. patent application Ser. Nos. 5,731,434, 5,622,971, and 5,767,144, the specifications of which are hereby incorporated by reference into this application, and commonly owned PCT applications WO/06095, published Feb. 29, 1996; WO 97/30045, published Aug. 21, 1997; and WO 99/06397, published Feb. 11, 1999.

A most preferred ET receptor antagonist useful for the practice of this invention is (2R,3R,4S)-2-(4-methoxyphenyl)-4-(1,3-benzodioxol-5-yl)-1-(N,N-di(n-butyl) aminocarbonylmethyl)pyrrolidine-3-carboxylic, also known as atrasentan.

### Determination of Health-Related Quality-Adjusted Time to Progression

The health-related QATTP model used for the practice of this invention expresses progression-free time as an equally preferable amount of time spent in full health. This is achieved by using patient-reported health-related QoL, as measured for the duration of observation or progression-free interval, to weight progression-free time. These data were collected from a plurality of randomized patients having hormone refractory prostate cancer (HRPCa) with the following validated instruments: the European Organization for Research and Treatment of Cancer Quality of Life Questionnaire (EORTC QLQ-30) and the Functional Assessment of Cancer Therapy (FACT-G) and its prostate cancer-specific module (FACT-P).

Patients received treatment with either 10 mg (N=89) or 2.5 mg (N=95) of atrasentan or placebo (N=104) until experiencing a clinical event indicative of disease progression as defined by palliative opiate treatment of new bone or visceral pain, palliative radiation treatment of new bone pain, or new tumor growth symptoms requiring intervention or treatment with chemotherapy.

Patient-reported health-related QoL data were collected with the EORTC QLQ-30 and the FACT-G and FACT-P, both of which were administered at baseline, at six week intervals and at the patient's final visit.

A patient's transformed domain score and total score from both the EORTC QLQ-30 and FACT were used to weight the time-to-progression outcome data. Transformed domain scores ranged between 0 and 1, so the reported health-related QATTP outcome was never larger than actual time to progression. The methods used for converting domain scores to weight adjustments are shown in Table 1.

TABLE 1
Transformation of Health-Related QoL Instrument Domain Scores to Weighted
Adjustments

| | DOMAIN SCORE | CONVERSION METHOD |
|---|---|---|
| INSTRUMENT AND DOMAIN NAME | RANGE | TO UNIT SCALE |
| EORTC Physical Functioning, | 0-100 | Domain Score/100 |
| Emotional Functioning, Role | | |
| Functioning, Social Functioning, | | |
| Cognitive Functioning, and Global | | |
| Score^{a} | | |
| EORTC Pain, Fatigue, Nausea and | 0-100 | 1 − (Domain Score/100) |
| Vomiting, Appetite Loss, Dyspnea, | | |
| Sleep Disturbance, Diarrhea, | | |
| Constipation^{b} | | |
| FACT Physical, Social/Family. | 0-28 | Domain Score-Lowest Domain Score |
| Functional Well-being^{a} | | Domain Score Range |
| FACT Emotional Well-being^{a} | 0-20 | Domain Score-Lowest Domain Score |
| | | Domain Score Range |
| FACT-G^{a} | 0-112 | Domain Score-Lowest Domain Score |
| | | Domain Score Range |
| FACT-P^{a} | 0-48 | Domain Score-Lowest Domain Score |
| | | Domain Score Range |
| FACT-Total^{a} | 0-160 | Domain Score-Lowest Domain Score |
| | | Domain Score Range |

^{a}A higher score means a better health-related QoL. A higher transformed score means improved health-related QoL.
^{b}A higher score means a worse health-related QoL. A higher transformed score means improved symptoms.

Possible scores for the fourteen EORTC domains each range between 1 and 100. For six domains (physical, emotional, role, social, and cognitive functioning and global score), a higher score means a better health-related QoL. These six scores were transformed to weights by dividing the patient-reported scores by 100.

For the remaining eight EORTC domains, a higher score indicates worse symptoms (a worse health-related QoL). These domains are pain, fatigue, nausea and vomiting, appetite loss, dysnepa, sleep disturbance, diarrhea, and constipation. These eight scores were converted to weight adjustments by dividing them by 100 and subtracting the result from the integer 1 to provide consistent directionality of response. FACT domain scores were converted to weights using a linear affine transformation suggested in SF-36 Health Survey Manual and Interpretation Guide.

Each patient's health-related QATTP was computed as the sum of the health-related QoL weights and the duration for which that patient experienced that health-related QoL.

If a patient experienced a clinical event between two health-related QoL assessments, the set of health-related QoL domain scores immediately prior to the event were carried forward to the time of the clinical event. The mean and median health-related QATTP outcomes were then estimated using Kaplan-Meier product limit methodology (Journal of the American Statistical Association, vol.53, 1958, pp 457-481). The area under a Kaplan-Meier survival curve conveys an estimated mean health-related QATTP. This analysis was applied to both intent-to-treat (ITT) and per protocol population data. All health-related QATTP comparisons between atrasentan and placebo treatment groups were based on log-rang test statistical significance at an α of 0.05.

Results of the Kaplan-Meier product limit survival method analysis are reported in Table 2 (Intent to Treat) and Table 3 (Per Protocol Population). Mean and median health-related QATTP are shown by treatment arm. Log-rank tests comparing the differences between treatment groups are also reported.

The Kaplan-Meier product limit method can provide biased results if study data are obtained under certain conditions such as staggered entry of subjects into the study and/or incomplete follow-up (Biometrics. 1989; 5:781-795). Thus, a second study was implemented to verify that the conclusions derived from the Kaplan-Meier would remain robust to the length of follow-up. The assumption was that all patients were followed for one year. Patients who discontinued from the study prior to one year of observation had their last observation for all health-related QoL domains carried forward through the remainder of the year. Similarly, patients who had not completed one year of observation had their health-related QoL data carried forward through one year. If the patient experienced a clinical event within the one year period, the last observation was not carried forward. The Area under the Curve (AUC) value for each domain was computed by multiplying the health-related QoL domain score by the respective duration of that score. Finally, AUC values were aggregated across all subjects within each respective treatment group (atrasentan (10 mg), atrasentan (2.5 mg), and placebo). Aggregated AUC values for each domain were compared for differences between treatment groups using a t-test.

TABLE 2
QATTP (Intent to Treat Data)

| | | | | | | | P-Value | | |
|---|---|---|---|---|---|---|---|---|---|
| | | | | | | | Log Rank Comparison | | |
| | QATTP | | | | | | Health-Related | | |
| | Median (days) | | | Mean (days) | | | QATTP^{a} | | |
| | At. | At. | | At. | At. | | 10 mg | | |
| QoL Domain Score Used for | (10 | (2 5 | | (10 | (2.5 | | vs. 2.5 | 10 mg | 2.5 mg |
| Adjusting Time-to Progression | mg) | mg) | Pl. | mg) | mg) | Pl. | mg | vs. Pl.^{a} | vs. Pl.^{a} |
| EORTC Physical Functioning | 119 | 118 | 137 | 164 | 172 | 86 | 0.796 | 0.091 | 0.118 |
| EORTC Emotional Functioning | 125 | 133 | 147 | 167 | 177 | 115 | 0.770 | 0.239 | 0.225 |
| EORTC Role Functioning | 123 | 128 | 145 | 180 | 176 | 106 | 0.863 | 0.160 | 0.205 |
| EORTC Social Functioning | 135 | 142 | 151 | 190 | 184 | 112 | 0.722 | 0.106 | 0.209 |
| EORTC Cognitive Functioning | 138 | 151 | 151 | 163 | 180 | 106 | 0.920 | 0.214 | 0.246 |
| EORTC Pain | 127 | 133 | 139 | 172 | 179 | 106 | 0.753 | 0.119 | 0.170 |
| EORTC Fatigue | 104 | 109 | 134 | 153 | 165 | 97 | 0.847 | 0.169 | 0.222 |
| EORTC Nausea & Vomiting | 156 | 162 | 169 | 201 | 195 | 129 | 0.655 | 0.165 | 0.323 |
| EORTC Appetite Loss | 146 | 151 | 161 | 175 | 186 | 118 | 0.616 | 0.157 | 0.309 |
| EORTC Dyspnea | 123 | 141 | 153 | 177 | 173 | 101 | 0.628 | 0.200 | 0.425 |
| EORTC Sleep Disturbance | 123 | 127 | 143 | 158 | 172 | 102 | 0.933 | 0.253 | 0.249 |
| EORTC Diarrhea | 176 | 178 | 169 | 185 | 198 | 129 | 0.733 | 0.201 | 0.270 |
| EORTC Constipation | 132 | 142 | 153 | 189 | 180 | 127 | 0.841 | 0.214 | 0.297 |
| EORTC Global Score | 103 | 104 | 119 | 141 | 159 | 93 | 0.928 | 0.245 | 0.242 |
| FACT Physical Well Being | 127 | 135 | 151 | 184 | 181 | 117 | 0.736 | 0.176 | 0.283 |
| FACT Emotional Well Being | 127 | 133 | 146 | 163 | 180 | 112 | 0.888 | 0.251 | 0.260 |
| FACT Social/Family Well Being | 121 | 126 | 141 | 147 | 160 | 104 | 0.771 | 0.277 | 0.380 |
| FACT Functional Well Being | 98 | 111 | 134 | 140 | 161 | 98 | 0.943 | 0.382 | 0.318 |
| FACT-G | 123 | 129 | 143 | 160 | 172 | 112 | 0.835 | 0.208 | 0.290 |
| FACT-P | 107 | 115 | 122 | 135 | 152 | 87 | 0.770 | 0.202 | 0.273 |
| FACT Total | 117 | 125 | 137 | 152 | 166 | 105 | 0.796 | 0.191 | 0.279 |

^{a}P-Values from Kaplan-Meier log-rank test of differences in health-related QATTP curves.
At. is atrasentan.
Pl. is placebo.

As shown in Table 2, the mean and median health-related QATTP showed no statistically significant differences between atrasentan treatment groups and placebo. Further, there were no statistically significant differences between the atrasentan treatment groups.

TABLE 3
QATTP (Per Protocol Data)

| | | | | | | | P-Value | | |
|---|---|---|---|---|---|---|---|---|---|
| | | | | | | | Log Rank Comparison | | |
| | QATTP | | | | | | Health-Related | | |
| | Median (days) | | | Mean (days) | | | QATTP^{a} | | |
| | At. | At. | | At. | At. | | 10 mg | | |
| QoL Domain Score Used for | (10 | (2 5 | | (10 | (2.5 | | vs. 2.5 | 10 mg | 2.5 mg |
| Adjusting Time-to Progression | mg) | mg) | Pl. | mg) | mg) | Pl. | mg^{a} | vs. Pl.^{a} | vs. Pl.^{a} |
| EORTC Physical Functioning | 127 | 124 | 85 | 168 | 181 | 128 | 0.932 | 0.019* | 0.014* |
| EORTC Emotional Functioning | 134 | 143 | 110 | 169 | 186 | 135 | 0.856 | 0.038* | 0.021* |
| EORTC Role Functioning | 128 | 142 | 100 | 187 | 185 | 134 | 0.944 | 0.030* | 0.024* |
| EORTC Social Functioning | 141 | 156 | 106 | 196 | 192 | 140 | 0.811 | 0.017* | 0.025* |
| EORTC Cognitive Functioning | 144 | 156 | 106 | 159 | 190 | 142 | 0.944 | 0.040* | 0.031* |
| EORTC Pain | 137 | 142 | 104 | 178 | 188 | 130 | 0.864 | 0.021* | 0.021* |
| EORTC Fatigue | 111 | 127 | 97 | 158 | 174 | 125 | 0.980 | 0.042* | 0.032* |
| EORTC Nausea & Vomiting | 168 | 178 | 127 | 207 | 206 | 158 | 0.792 | 0.029* | 0.042* |
| EORTC Appetite Loss | 146 | 162 | 118 | 179 | 196 | 150 | 0.731 | 0.027* | 0.040* |
| EORTC Dyspnea | 132 | 142 | 98 | 182 | 180 | 145 | 0.738 | 0.060 | 0.119 |
| EORTC Sleep Disturbance | 127 | 137 | 101 | 162 | 179 | 131 | 0.960 | 0.043* | 0.030* |
| EORTC Diarrhea | 184 | 184 | 127 | 188 | 209 | 159 | 0.892 | 0.37* | 0.029* |
| EORTC Constipation | 141 | 151 | 120 | 198 | 190 | 145 | 0.935 | 0.049* | 0.047* |
| EORTC Global Score | 106 | 124 | 90 | 145 | 167 | 113 | 0.975 | 0.057 | 0.038* |
| FACT Physical Well Being | 130 | 148 | 112 | 190 | 191 | 142 | 0.835 | 0.036* | 0.039* |
| FACT Emotional Well Being | 131 | 147 | 107 | 168 | 188 | 137 | 0.995 | 0.053* | 0.035* |
| FACT Social/Family Well Being | 126 | 143 | 102 | 151 | 169 | 131 | 0.912 | 0.059 | 0.049* |
| FACT Functional Well Being | 113 | 111 | 96 | 144 | 168 | 126 | 0.960 | 0.119 | 0.056 |
| FACT-G | 130 | 143 | 105 | 165 | 180 | 135 | 0.957 | 0.047* | 0.040* |
| FACT-P | 111 | 117 | 81 | 139 | 160 | 115 | 0.909 | 0.038* | 0.035* |
| FACT Total | 127 | 135 | 102 | 157 | 174 | 129 | 922 | 0.040* | 0.035* |

^{a}P-Values from Kaplan-Meier log-rank test of differences in health-related QATTP curves.
*Significant at p < 0.05
At. is atrasentan.
Pl. is placebo.

As shown in Table 3, the health-related QATTP's were improved by treatment with atrasentan in both the 10 mg and 2.5 mg arms as compared to placebo in the per protocol analysis. As shown in Table 2, weighted health-related QATTP in the 10 mg A group were significantly longer (p<0.05) than in the placebo group for all health-related QoL domains except for “dyspnea,” “global score,” “emotional well-being,” “social well-being,” and functional well-being.” In four of these domains, (dyspnea, global score, emotional well-being, and social well-being), the trend favored treatment with 10 mg atrasentan over placebo (p<0.10).

Similar results were observed in comparison between groups treated with 2.5 mg of atrasentan over the placebo group. Treatment with 2.5 mg of atrasentan produced longer mean health-related QATTP than did placebo. Log rank tests showed these results to be statistically significant for all health-related QoL domains except “dyspnea” and “functional well-being.” However, there were no statistical differences noted between the atrasentan treatment groups for any health-related QoL domain.

The (AUC) analysis results were consistent with the health-related QATTP analyses in Tables 2 and 3. For the Intent to Treat population (Table 2), atrasentan treatment and placebo groups showed no statistical differences. The AUC analysis of the per-protocol population showed strong trends in favor of the atrasentan treatment groups in every health-related QoL domain score when compared to placebo. The responses in the 10 mg and 2.5 mg groups were not statistically differentiable.

The AUC for the health-related QoL domain scores for “physical functioning,” “social functioning,” and “pain” were significantly (p<0.05) longer for atrasentan than placebo. Similarly, the 2.5 mg atrasentan group showed significantly improved AUC results than the placebo group, except for “dyspnea, “social/family,” and “functional well-being,” and FACT-P domain scores.

The results in the Per Protocol analysis (Table 3) demonstrated that 10 mg and 2.5 mg of atrasentan delayed the time to disease progression compared to placebo. For these subjects, 10 mg and 2.5 mg of atrasentan also delayed the time to prostate-specific antigen (PSA) progression and attenuated increases in markers of bone metabolism. These analyses, however, did not take into account the impact of therapy on the patients' perceived health status.

To address these issues, this analysis examined the effects of 10 mg and 2.5 mg of atrasentan taking into account not just time to disease progression but also the patients' health-related QoL during this time. This was achieved by weighting the time-to-progression outcomes from the PSA progression and bone metabolism studies using patient-reported health-related QoL scores. Patient-reported health-related QoL data has validity for two reasons: the perception of health is stated by the patient directly, and multidimensional health-related QoL instruments provide a more complete and balanced assessment of patients' health status. It was found that after adjusting for health-related QoL effects, both 10 mg and 2.5 mg atrasentan therapies offered longer health-related QATTP over placebo in the per protocol population. These gains in the health-related QATTP were robust over a wide range of health-related QoL domain weighting and were consistently observed using the EORTC and FACT as the two health-related QoL instruments. For the intent-to-treat population, there were no statistically significant differences in the QATTP across treatment groups. This finding is consistent with the fact that, for the intent-to-treat population, no statistically significant differences were observed in either the time to disease or PSA progression across treatment groups.

Additional AUC analyses showed that after adjusting for possible bias induced by unequal lengths of follow-up and the staggered entry of subjects, the findings produced by the Kaplan-Meier methods were confirmed.

Thus, the QATTP analysis shows that subjects treated in the per protocol population treated with either 10 mg or 2.5 mg of atrasentan realized net clinical benefits and improved health status from the extension in time to disease progression.

What is claimed is:

1. A method for favorably modulating the health-related quality-adjusted time-to-progression of a patient with prostate cancer comprising administering to the patient a therapeutically effective amount of an endothelin receptor antagonist.

2. The method of claim 1 in which the prostate cancer is hormone refractory prostate cancer.

3. The method of claim 1 in which the therapeutically effective amount of the endothelin receptor antagonist is between about 0.01 mg per day to about 100 mg per day.

4. The method of claim 3 in which the therapeutically effective amount of the endothelin receptor antagonist is between about 1 mg per day to about 25 mg per day.

5. The method of claim 4 in which the therapeutically effective amount of the endothelin receptor antagonist is about 2.5 mg per day.

6. The method of claim 4 in which the therapeutically effective amount is about 10 mg per day.

7. The method of claim 1 in which the endothelin antagonist is atrasentan.
